(19) United States
(12) Patent Application Publication
(10) Pub. No.: US 2015/0125838 A1
(43) Pub. Date: May 7, 2015

(54) Methods and Systems for Hands-On Development Therapy

(71) Applicant: Caroline Pack, Roosevelt, UT (US)

(72) Inventor: Caroline Pack, Roosevelt, UT (US)

(21) Appl. No.: 14/531,851

(22) Filed: Nov. 3, 2014

Related U.S. Application Data
(60) Provisional application No. 61/898,688, filed on Nov. 1, 2013.

(51) Int. Cl. G09B 19/00; A61B 5/16

(52) U.S. Cl. CPC G09B 19/003; A61B 5/16
USPC 434/258

(57) ABSTRACT

Methods and systems of providing hands-on developmental therapy to a subject by a care provider are disclosed. The method can include providing a subject to receive hands-on therapy, assessing the developmental needs of the subject, selecting a level corresponding to the developmental needs of the subject, providing the subject with therapeutic tools and activities corresponding to the therapeutic tools, and guiding the subject through the hands-on therapy corresponding to the selected level. The levels include activities of increasing difficulty with the subject using the same therapeutic tools for each level. The levels can also include a sensory level with sensory tools and activities to improve the sensory system of the subject. The methods and systems of providing hands-on developmental therapy can improve a subject's fine motor skills, strength, and sensory skills.

### CROSS-REFERENCE TO RELATED APPLICATIONS

This application claims priority to U.S. Provisional Patent Application No. 61/898,688 filed Nov. 1, 2013, titled Methods and Systems for Hands-on Development Therapy. The disclosure of each of the applications to which the present application claims priority are incorporated by reference.

### BACKGROUND

A person's sensory system enables the person to interpret and/or interact with his or her environment. The same sensory system impacts a person's ability to learn basic skills, to develop, or to participate in recreation. People with disabilities and/or other developmental disorders often have compromised sensory systems and require therapy to aid in the development of their sensory systems. This requirement for therapy is most acute in children with disabilities and/or other developmental disorders. In many cases, people that are affected by these disabilities and/or other developmental disorders are able to receive therapy to overcome these developmental problems. Therapy can involve visiting a therapist for therapy sessions. During these therapy sessions, the affected person is able to engage in activities to develop target skills. However, between these therapeutic visits there are limited opportunities for the affected person to continue working on activities to develop these target skills. Moreover, the required therapeutic tools to carry out these activities are lacking in the affected person's home setting. There is a need for a variety of easily accessible and interchangeable therapeutic tools to aid in an affected person's therapeutic development between therapeutic visits. These therapeutic tools are needed to help affected persons maintain the focused attention necessary to acquire the targeted skills. These therapeutic tools are also needed for affected persons not being personally treated by a therapist.

### BRIEF SUMMARY

Methods and systems for hands-on development therapy are disclosed. In some embodiments, the methods and systems can comprise a three-tiered organized therapeutic kit or toolbox with tools and activities to develop, improve or regain targeted skills. In other embodiments, the kit may comprise purposefully selected activities that may be used separately or in combination, to aid a subject's therapy.

In some embodiments, the methods and systems can comprise a method of providing hands-on developmental therapy to a subject by a care provider with the method comprising providing a subject to receive hands-on therapy, providing the subject with a container comprising a plurality of therapeutic tools, wherein the therapeutic tools are grouped into three levels, selecting a level corresponding to a hands-on therapy that is appropriate for the subject, removing from the container the plurality of therapeutic tools, and guiding the subject through the hands-on therapy corresponding to the selected level. In other embodiments, the subject can suffer from a developmental disorder. In yet other embodiments, the developmental disorder can include one or more of Down's syndrome, autism, ADD/ADHD, dyslexia, developmental delay, Fragile-X syndrome, sensory processing disorder, sensory integration dysfunction, learning disability, cognitive disability, or cerebral palsy.

In some embodiments, the hands-on therapy can be part of a therapy program administered by a therapist. In other embodiments, the therapist can include one or more of a physical therapist, an occupational therapist, or a speech and language therapist. In yet other embodiments, the hands-on therapy is provided in the subject's home between visits to a therapist.

In some embodiments, the method can comprise three levels, with each level comprising activities of increasing difficulty and with the subject using the same plurality of therapeutic tools for each level. In other embodiments, the method can further comprise a sensory level, with the sensory level comprising therapeutic tools and activities configured to improve the sensory system of the subject. In some embodiments, more than one level is selected. In other embodiments, the three levels respectively correspond to hands-on therapy configured to improve fine motor skills, to improve strength, or to improve sensory skills. In yet other embodiments, the hands-on therapy comprises the subject interacting with the therapeutic tools in developmental activities corresponding to daily living activities. In some embodiments, the hands-on therapy comprises the subject interacting with the therapeutic tools in developmental activities corresponding to academic activities. In other embodiments, the hands-on therapy comprises the subject interacting with the therapeutic tools in developmental activities corresponding to writing activities. In yet other embodiments, the hands-on therapy further comprises the subject interacting with the container.

In some embodiments, a system of hands-on developmental therapy is disclosed. In other embodiments, a system of hands-on developmental therapy can comprise a subject requiring a hands-on therapy, a container comprising a plurality of therapeutic tools, with the therapeutic tools are grouped into three levels, and a care provider. The care provider can select the level corresponding to the hands-on therapy that is appropriate for the subject, can remove from the container the plurality of therapeutic tools, and can guide the subject through the hands-on therapy corresponding to the selected level. In yet other embodiments, the subject can suffer from a developmental disorder. In some embodiments, the developmental disorder can include one or more of Down's syndrome, autism, ADD/ADHD, dyslexia, developmental delay, Fragile-X syndrome, sensory processing disorder, sensory integration dysfunction, learning disability, cognitive disability, or cerebral palsy. In other embodiments, the system can comprise three levels respectively corresponding to hands-on therapy configured to improve fine motor skills, to improve strength, or to improve sensory skills.

In some embodiments, a kit for providing hands-on developmental therapy to a subject by a care provider is disclosed. In other embodiments, the kit can comprise a container comprising three tiers and a plurality of therapeutic tools, the therapeutic tools grouped into three levels. A care provider can select the level corresponding to a hands-on therapy that is appropriate for a subject, can remove from the container the plurality of therapeutic tools corresponding to the selected level, and can guide the subject through the hands-on therapy corresponding to the plurality of therapeutic tools corresponding to the selected level. In yet other embodiments, the kit can comprise three levels respectively corresponding to hands-on therapy configured to improve fine motor skills, to improve strength, or to improve sensory skills. In some embodiments, subject can suffer from a developmental disorder. In other embodiments, the hands-on therapy can further comprise the subject interacting with the container.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A illustrates an ulnar palmar grasp;

FIG. 1B illustrates a palmar grasp;

FIG. 1C illustrates a radial palmar grasp;

FIG. 1D illustrates a radial digital grasp, also known as a tripod grasp;

FIG. 1E illustrates a scissors grasp;

FIG. 1F illustrates a pincer grasp;

FIG. 2A illustrates a perspective view of a container;

FIG. 2B illustrates a front view of a container;

FIG. 3A is a depiction of beads;

FIG. 3B is a depiction of bendeez;

FIG. 3C is a depiction of block crayons;

FIG. 3D is a depiction of centimeter cubes;

FIG. 3E is a depiction of erasers;

FIG. 3F is a depiction of exercise tubing;

FIG. 3G is a depiction of lacing;

FIG. 3H is a depiction of magnet balls;

FIG. 3I is a depiction of magnet discs;

FIG. 3J is a depiction of magnet rings;

FIG. 3K is a depiction of a magnet wand;

FIG. 3L is a depiction of marbles;

FIG. 3M is a depiction of nuts and bolts;

FIG. 3N is a depiction of pegs/pegboard;

FIG. 3O is a depiction of poms;

FIG. 3P is a depiction of pop tubes;

FIG. 3Q is a depiction of rainbow caps;

FIG. 3R is a depiction of rainbow links;

FIG. 3S is a depiction of a sand timer;

FIG. 3T is a depiction of scizzors;

FIG. 3U is a depiction of stamps;

FIG. 3V is a depiction of stringing rings;

FIG. 3W is a depiction of theraputty;

FIG. 3X is a depiction of tongs;

FIG. 3Y is a depiction of tracing cards;

FIG. 3Z is a depiction of a wire wizard;

FIG. 3AA is a depiction of wooden cubes;

FIG. 3AB is a depiction of a slotted cap;

FIG. 3AC is a depiction of stringing balls in a tube container;

FIG. 4A is a depiction of a blow fish;

FIG. 4B is a depiction of an egg shaker;

FIG. 4C is a depiction of a finger spring;

FIG. 4D is a depiction of a flying frog;

FIG. 4E is a depiction of a gel exercise ball;

FIG. 4F is a depiction of a hand massager/top;

FIG. 4G is a depiction of a jiggler;

FIG. 4H is a depiction of a jumping frog;

FIG. 4I is a depiction of a klixx;

FIG. 4J is a depiction of a liquid timer;

FIG. 4K is a depiction of a magic wire loop;

FIG. 4L is a depiction of a net squeeze toy;

FIG. 4M is a depiction of a smiley stress ball;

FIG. 4N is a depiction of a spike ball/finger massager;

FIG. 4O is a depiction of a gel bead pack;

FIG. 4P is a depiction of a gel squeeze toy; and

FIG. 5 illustrates a method for hands-on development therapy.

### DETAILED DESCRIPTION OF THE INVENTION

The present application discloses methods and systems for hand-on development therapy. In some embodiments, the methods and systems can comprise a three-tiered organized therapeutic kit or toolbox with therapeutic tools and activities to develop, improve or regain targeted skills. In other embodiments, the kit may comprise purposefully selected activities that may be used separately or in combination, to aid a subject's therapy. In yet other embodiments, the methods and systems can comprise therapeutic activities corresponding to the therapeutic tools to aid in developing targeted skills. In some embodiments, the methods and systems can comprise therapeutic activities that are divided into levels that correspond to increasing difficulty and/or correspond to different types of skills.

In some embodiments, the methods and systems are drawn to hands-on developmental therapies to aid a subject to develop targeted skills. A subject can include any person that needs to develop targeted skills. In other embodiments, subjects can include children, adults and the elderly. In yet other embodiments, subjects can include any person with a developmental disability. The developmental disability may be the result of birth, injury and/or neurological condition. The targeted skills that each subject may need to acquire can depend on the individual circumstances of the subject and the nature of injury of developmental disability that affects the subject. Subjects with certain disabilities, injuries, and/or neurological conditions often have common needs and can require therapy to develop common targeted skills Understanding each subject's developmental disabilities and/or injuries can assist in selecting a hands-on developmental therapy that will be effective for the subject. In some embodiments, subjects with the developmental disabilities described below can require common targeted skills.

In some embodiments, subjects with Down's syndrome can suffer from weak muscle tone, weak fine motor skills, severe mental impairment, and flattened facial profile. In subjects with Down's syndrome, hands-on developmental therapy can improve muscle tone and movements, help achieve developmental milestones, and prevent future motor problems that accompany low muscle tone. Hands-on developmental therapy can be configured to meet these goals and to address developmental difficulties.

In some embodiments, subjects with autism can suffer from emotional detachment, decreased sensory processing, and impaired communication. In subjects with autism, hands-on therapy can assist the subject in transitioning to new activities, developing motor skills, manipulating objects, learning play skills, responding to touch and stimuli, improving attention span, interacting with others, learning posture, and improving balance. Hands-on developmental therapy can be configured to meet these goals and to address developmental difficulties associated with autism. Hands-on therapy can be adapted to subjects of any age that are trying to be more independent and function in his or her environment.

In some embodiments, subjects with ADD/ADHD (Attention Deficit Disorder/Attention Deficit Hyperactivity Disorder) can suffer from an inability to pay attention, inability to concentrate, inability to restrain impulsive behavior, difficulty with academic tasks, difficulty with staying calm and focused. In other embodiments, subjects with ADD/ADHD can suffer extreme difficulty in following directions and can find tasks to be boring or frustrating. In yet other embodiments, these problems can interfere with the subject's ability to perform tasks at home, to hear or read instructions, to complete school assignments, and to participate in games and activities. Hands-on developmental therapy can be configured to address these difficulties associated with ADD/ADHD. In some embodiments, hands-on developmental therapy can comprise timers, sequencing, patterning, and memory games that can assist to overcome these difficulties. In some embodiments, the hands-on developmental therapy can assist subjects with ADD/HD to learn to follow directions, place objects and ideas together, follow patterns to assist with academic learning, and function in life successfully in regard to independent living.

In some embodiments, subjects with dyslexia can suffer from a variety of reading disorders associated with impairment of the ability to interpret spatial relationships and/or inability to integrate visual and auditory information. Hands-on developmental therapy can be configured to address difficulties with tasks that require fine and gross motor coordination. These tasks can include using scissors, writing, self-care tasks, tying shoelaces, hand-eye coordination, catching a ball, and copying from a chalkboard. In other embodiments, subjects can include adults with dyslexia. In yet other embodiments, hands-on developmental therapy can be configured to assist adult subjects with dyslexia to learn to write checks, learn adaptions for spatial relationships through sensory activities, and other skills required for independence.

In some embodiments, subjects with developmental delay can suffer from significant delay in achieving developmental milestones at the expected times. In other embodiments, developmental delay can include delay in developing fine motor skills, gross motor skills, cognitive skills, behavioral skills, emotional skills and social skills. In yet other embodiments, hands-on developmental therapy can be configured to assist subjects with developmental delay to achieve developmental milestones. Reaching developmental milestones can be important because developmental skills are typically learned in a sequential manner. In some embodiments, the therapeutic tools can assist the subject to learn in a sequential manner. In other embodiments, the hands-on developmental therapy can be divided into three levels of progression to improve overall function.

In some embodiments, subjects with Fragile X syndrome can suffer from intellectual disabilities. Difficulty performing self care tasks: grooming, brushing teeth, getting dressed, etc. In other embodiments, subjects with Fragile X can suffer from a large range of issues including sensory problems, social issues, behavioral issues, and communication issues. Hands-on developmental therapy can comprise sensory therapeutic tools to provide a broad range of sensory experiences. In yet other embodiments, sensory therapeutic tools can include objects that range from soft to hard, from smooth to rough, from firm for strength to soft for a more intense sensory experience, from vibration used for writing and alerting the sensory system to lava timers to calm and relax. These sensory tools can assist subjects with Fragile X syndrome to overcome these issues and provide them with tools to help with every day activities.

In some embodiments, subjects can suffer from Sensory Processing Disorder or Sensory Integration Dysfunction. In other embodiments, subjects with sensory processing disorder can have difficulty receiving and responding to information that comes in through the senses. In yet other embodiments, subjects with sensory processing disorder can suffer from oversensitivity to the environment, inability to know where their body is in space, difficulty engaging in communication or play, and uncoordinated movements. Hands-on developmental therapy can be configured to assist subjects with sensory processing disorder through the use of sensory tools and an array of activities which can engage the individual to work on affected development areas.

In some embodiments, subjects with a learning disability can suffer from a significant delay in social, physical, emotional, cognitive, and/or behavioral development. Hands-on developmental therapy can be configured to addresses goals and functions for a subject's current level of development. Hands-on developmental therapy can also provide activities to assist in gaining and progressing in fine motor skills, coordination, emotional behaviors, handwriting, sequencing, and patterning.

In some embodiments, subjects with cognitive disability can suffer from developmental delay, physical difficulties, inability to retain information, and loss of memory. In other embodiments, for subjects with cognitive disability, hands-on developmental therapy can be configured to address fine and gross motor tasks, adaptation to environment, memory activities, and consistent scheduling for establishing routines. Hands-on developmental therapy can assist with skills and tasks to overcome these difficulties. In yet other embodiments, subjects with cognitive disabilities can be provided hands-on developmental therapy comprising using patterning activities, organizing tools into categories, addressing object permanence, using sensory toys to stimulate cognitive function and alertness, and throwing and catching to address coordination. In some embodiments, for subject suffering from a cognitive disability, activities can be broken down into smaller components.

In some embodiments, subjects with cerebral palsy can suffer from an inability to control motor function, particularly in muscle control and coordination. In other embodiments, subjects with cerebral palsy can suffer from different developmental difficulties depending on which areas of the brain may have been damaged. In yet other embodiments, subject with cerebral palsy can suffer from difficulty in gait and mobility, involuntary movement, muscle tightness, decreased sensation and perception, difficulty with sight, hearing, and speech, and seizures. In some embodiments, hands-on developmental therapy can be configured to assist with grasp and release, handwriting, improving gross motor skills, catching and throwing, activities of daily living, removing screw-top lids (such as a toothpaste lid) and learning positive ways to calm his or her sensory system.

In some embodiments, a subject may have mental disabilities or neurological disorders. In other embodiments, a subject may have mental disabilities or neurological disorders in addition to a developmental disability that may require the subject to learn a certain set of targeted skills Hands-on developmental therapy can assist with skills and tasks to overcome these difficulties, such as sequencing, memory, visual tracking, and patterning, etc. In some embodiments, a subject may also have a mobility impairment that can determine additional targeted skills to be addressed by the hands-on developmental therapy. In other embodiments, a subject may have an injury that causes developmental difficulties.

In some embodiments, a subject may suffer from a disorder related to a government mandate. In other embodiments, the government mandate can require that a subject suffering from certain disorders receive developmental therapy. In yet other embodiments, the government mandate can apply to subjects with autism. In some embodiments, the government mandate can provide government funding to reimburse subjects for costs of developmental therapy. In other embodiments, the methods and systems of this disclosure satisfy one or more requirements covered by a government mandate.

In some embodiments, the hands-on developmental therapy can be administered to a subject by a care provider. In other embodiments, the care provider can be a parent or a guardian. In yet other embodiments, the care provider can be a grandparent or other relative. In some embodiments, the care provider can be a licensed therapist. In other embodiments, the care provider can be a health care worker. In yet other embodiments, the care provider can be a teacher. In some embodiments, the care provider can be a child care provider.

In some embodiments, the hands-on developmental therapy can be configured for use in the following settings: schools, therapy departments, care centers, rehabilitation, hospitals, home health, home schools, and other similar settings.

In some embodiments, the methods and systems are drawn to hands-on developmental therapies to aid a subject develop targeted skills. In other embodiments, these targeted skills may comprise fine motor, strength, and sensory skills. In yet other embodiments, these targeted skills can comprise bilateral coordination, motor planning, pre-writing skills, distal finger control, proximal stability, thumb opposition, visual motor, visual memory, mid-line activities, attention to task, eye-hand coordination, fine motor strength, and sensory awareness. In some embodiments, the targeted skills can comprise finger and hand strength.

In some embodiments, the methods and systems can comprise assessing the subject and adapting a hands-on therapy to the subject. In other embodiments, assessing the subject can comprise screening by a health care worker. In yet other embodiments, assessing the subject can comprise assessing the subject as part of well-child visits. In some embodiments, assessing the subject can comprise screening for developmental delays and/or assessing the subject for the ability to reach developmental milestones. In other embodiments, assessing the subject can comprise assessing a subject's physical, mental, social, and emotional well-being. In yet other embodiments, assessing the subject can include screening for any of the conditions, disorders, or developmental conditions described above. In some embodiments, assessing the subject can comprise cataloging the physical, mental, social, and emotional needs of the subject. In other embodiments, assessing the subject can comprise screening the subject for fine motor, strength, and sensory skills. In other embodiments, assessing the subject can comprise screening the subject for bilateral coordination, motor planning, pre-writing skills, distal finger control, proximal stability, thumb opposition, visual motor, visual memory, mid-line activities, attention to task, eye-hand coordination, fine motor strength, and sensory awareness. In yet other embodiments, assessing the subject can comprise screening the subject for finger and hand strength. In some embodiments, assessing the subject can comprise ongoing screening to the subject and adaptation of the hands-on developmental therapy to the changing needs of the subject.

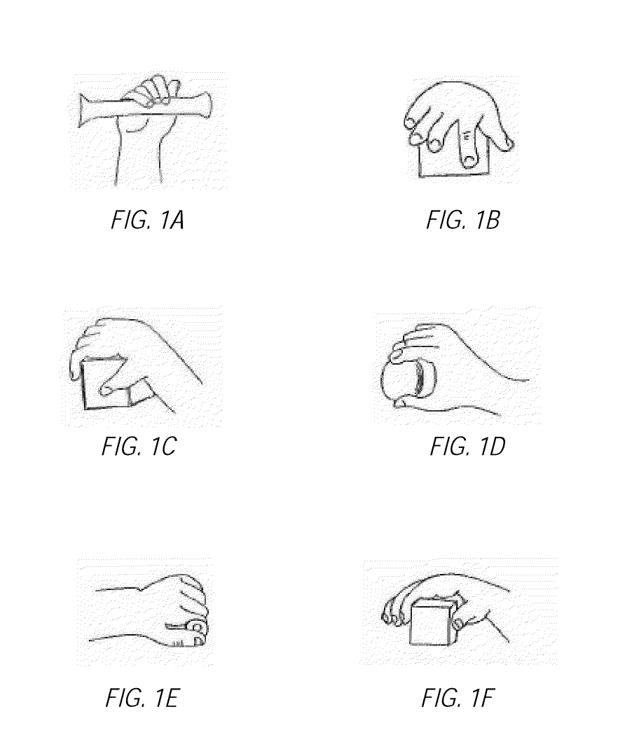

FIG. 1 illustrates some embodiments of hand movements that can be related to developing targeted skills FIG. 1A illustrates an ulnar palmar grasp. In some embodiments, an ulnar palmar grasp can comprise grasping an object in the center of the palm with the middle, ring, and little fingers. FIG. 1B illustrates a palmar grasp. In some embodiments, a palmar grasp can comprise grasping an object in the center of the palm using index, middle, and ring fingers. FIG. 1C illustrates a radial palmar grasp. In some embodiments, a radial palmar grasp can comprise grasping an object in the palm with thumb, index, and middle fingers. FIG. 1D illustrates a radial digital grasp or tripod grasp. In some embodiments, a radial digital grasp or tripod grasp can comprise grasping and object with thumb, index, and middle fingers with a space between object and palm. FIG. 1E illustrates a scissors grasp. In some embodiments, a scissors grasp can comprise grasping an object on the side of the index finger to hold small objects in a scissors grip. FIG. 1F illustrates a pincer grasp. In some embodiments, a pincer grasp can comprise grasping an object between thumb and the tip of the index finger. In other embodiments, the therapeutic tools, activities and levels of activities can be configured to aid the subject in developing one of more of the hand movements illustrated in FIG. 1.

In some embodiments, targeted skills can comprise one or more of manipulating buttons and snaps, screwing and unscrewing, opening and closing, using scissors, buckling and unbuckling, writing with proper grasp and legibility, holding small objects, isolating finger movements for musical instruments or electronics, doing puzzles, playing games with hand and finger control, zipping and unzipping, coloring and drawing, holding utensils, turning pages of a book, using coins and money, cooking and food preparation, maintaining and developing fine motor strength, and completing any task requiring precise finger and hand movements. In other embodiments, targeted skills can comprise activities of daily living (ADL). In yet other embodiments, ADLs can comprise eating, getting dressed, doing buttons and zippers, toileting, grooming, bathing, cooking, writing, sorting, patterning, and other similar activities. In some embodiments, targeted skills can comprise instrumental ADLs (IADL). In other embodiments, IADLs can comprise complex skills such as: using the telephone, doing essential housework, preparing meals, using kitchen equipment, using transportation, shopping, playing instruments, and recreational activities. In yet other embodiments, ADLs and IADLs can comprise extending fingers to reach for toothpaste, flexion of fingers and tripod grasp to hold caps, supination and pronation of wrist to twist caps off, coordinating fine motor and gross motor to squeeze paste while holding toothbrush in opposite hand, tolerating toothbrush and toothpaste in mouth, using a brushing motion and other similar activities.

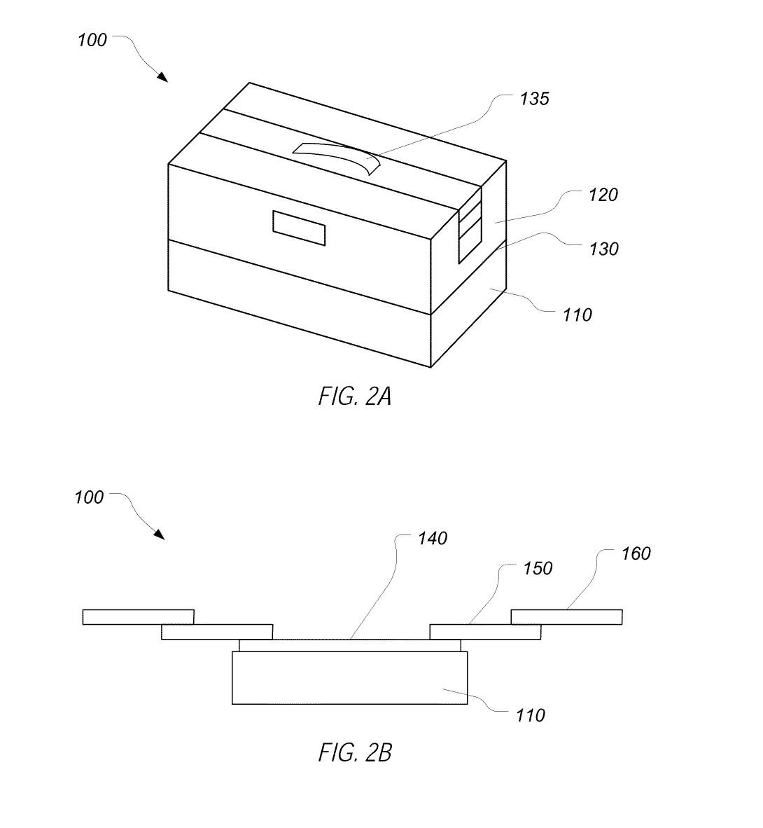

FIG. 2 illustrates embodiments of a container 100 that is configured to contain therapeutic tools for hands-on developmental therapy. FIG. 2A illustrates a perspective view of some embodiments of the container 100. In some embodiments, container 100 can comprise a lower portion 110 and an upper portion 120. In other embodiments, a zipper 130 can be configured to detachably connect three sides of the lower portion 110 and upper portion 120. The zipper 130 can be engaged to close the container 100. The zipper 130 can be opened to allow the upper portion to be folded back to open the container 100. In some embodiments, the container 100 further comprises a handle 135 configured to carry the closed container. FIG. 2B illustrates a front view of some embodiments of the container 100 in an open configuration. In some embodiments, the container 100 can comprise a lower compartment 140. In other embodiments the lower compartment 140 can comprise a first tier. In yet other embodiments, the container 100 can comprise additional compartments 150, 160. In other embodiments the additional compartments 150, 160 can be configured to collapse over the lower compartment 140 to allow the container 100 to be closed. In some embodiments, the additional compartments 150, 160 can comprise a second tier and a third tier. In other embodiments the first, second, and third tiers can be configured to contain different levels or classes of therapeutic tools.

In some embodiments, the hands-on developmental therapy can comprise therapeutic tools and corresponding activities. In other embodiments, the therapeutic tools and corresponding activities can be selected to aid in developing targeted skills. In yet other embodiments, the activities may be used alone. In some embodiments, some activities may be used in combination to create a composite activity. In other embodiments, the therapeutic tools and activities can give a subject access to therapeutic tools they can use on a daily basis. In yet other embodiments, the therapeutic tools can comprise tools that directly relate to activities of daily living. In some embodiments, the therapeutic tools can facilitate movements and muscles that can be related to self-care, academic achievement, and independent function in everyday life.

In some embodiments the activities can comprise therapeutic tools. In other embodiments, the therapeutic tools may be used in unison with each other to develop targeted skills. In yet other embodiments, the therapeutic tools can comprise sensory tool that can help subjects understand and interact with the world around them. In some embodiments, the use of the kit can motivate a subject to explore and encourage healthy processing for daily life. In other embodiments, the therapeutic tools and activities disclosed herein can delight, educate, and strengthen individuals' fine motor and sensory processing skills through hands on development.

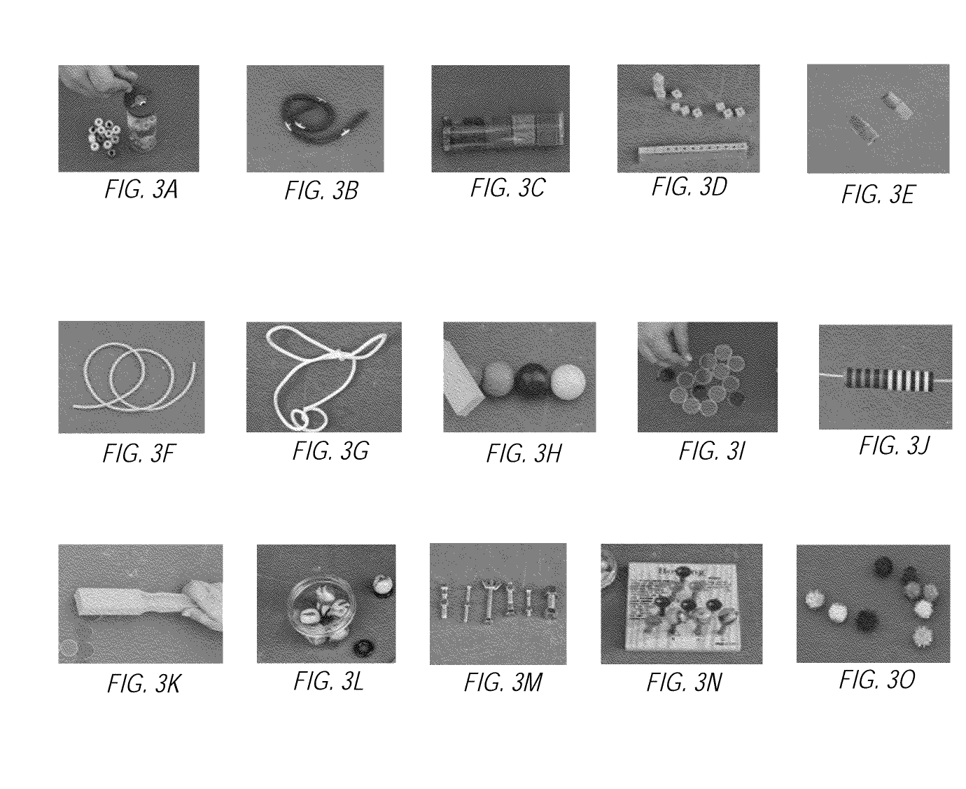

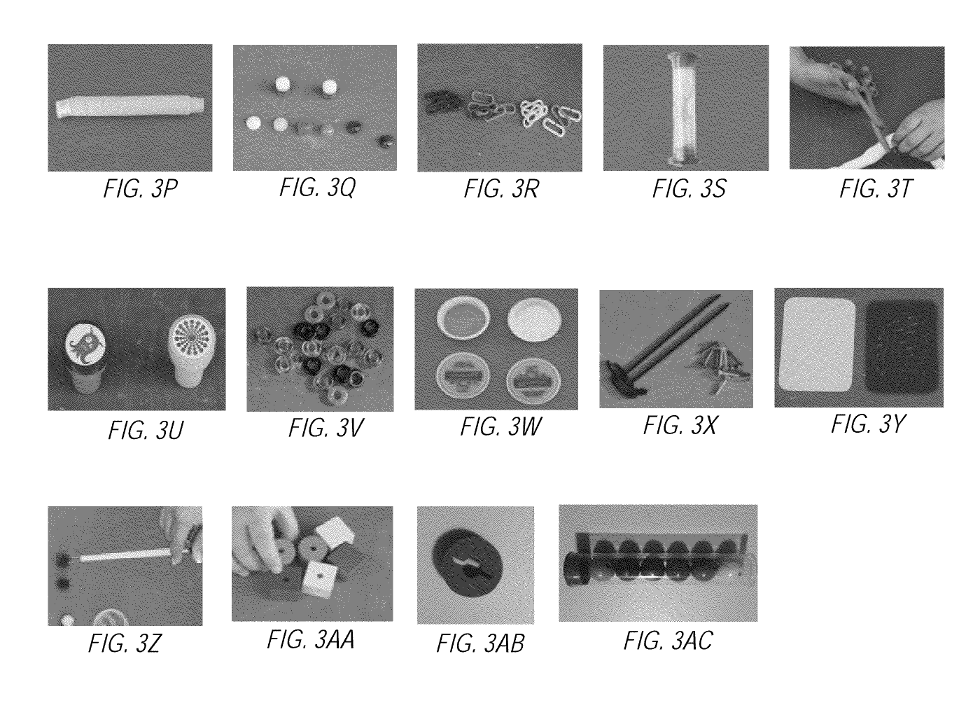

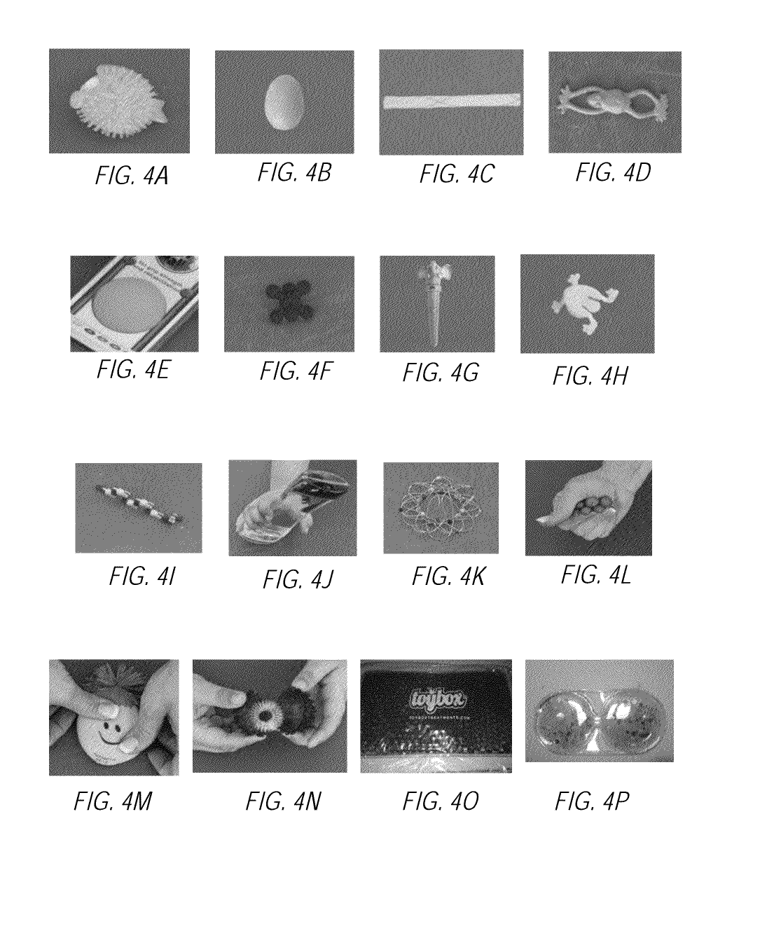

In some embodiments, the therapeutic tools and sensory tools can be contained in the container 100. Referring now to FIGS. 3A to 4P, certain embodiments of the hands-on developmental therapy can comprise over 150 purposefully selected therapeutic tools, sensory tools, and activities which can be used either separately or in combination to assist subjects with targeted skills. In some embodiments, the container 100 and the therapeutic and sensory tools can comprise a system for hands-on developmental therapy. In other embodiments, a system for hands-on developmental therapy can comprise 14 filled activity tubes, 3 twist containers complete with fine motor activities, 2 timers, 2 levels of theraputty, and one large bag of therapy tools including tongs, scissors, wire wizard, pencils, sensory tools, and hand strengthening tools. In yet other embodiments, the lower tier can include a variety of sensory balls, relaxing squeeze tools, finger and hand massagers, a special ball for hand strength and exercise, pop tubes for sensory and strength, and delightful tools for fine motor coordination.

In some embodiments, the therapeutic tools can comprise a set of tools comprising hands-on activities and tools that can be used in individually or in cooperation with other tools to help develop targeted skills. In other embodiments, therapeutic tools can comprise a variety of tools that can be used as the subject advances through different stages of development. In yet other embodiments, the therapeutic tools can further comprise an instruction booklet to guide the care provider in administering the hands-on developmental therapy. In some embodiments, the therapeutic tools can be easily accessible and interchangeable one with another. In other embodiments, the therapeutic tools can comprise a variety of sizes, textures, resistances, and activities to help develop targeted skills in a plurality of environments. In other embodiments, the therapeutic tools can comprise strength training tools that are easily adapted to different environments.

In some embodiments, the therapeutic tools can comprise writing tools focuses on specific hand functioning positions to assist in daily self-care tasks such as getting dressed, brushing teeth, food preparation, and any hand activity that requires a pincer or tripod grasp. In other embodiments, the therapeutic tools can comprise writing tools to transfer targeted skills used in daily self-care tasks to writing skills for academic purposes. In yet other embodiments, the therapeutic tools can comprise writing tools to assist the subject in developing the ability to hold a writing utensil and perform writing tasks. In some embodiments, the therapeutic tools can comprise tools that can be used in unison with each other. In other embodiments, the therapeutic tools can comprise tools that can be used together cooperatively to assist the subject in developmental therapy.

In some embodiment, the therapeutic tools can comprise groups of tools designed for a broad range of subjects with varying degrees of difficulty related to fine motor skills, sensory abilities, and strength. In other embodiments, the therapeutic tools can comprise: larger items to develop grasping patterns, adapting to open web space, beginning flexion/extension of hand and arms; medium sized items to improve development and manipulation of objects relating to holding a utensil, using a hair brush, and other activities and including items such as large buttons, zippers, etc. (magnet wand, tongs, wire wizard, bendeez); large twist and lock containers to mimic daily fine motor activities such as opening doorknobs, jars, lids, and similar items; medium to small items to develop movement from the palm to the fingers including items such as blocks, links, marbles, caps, and similar items; large, medium, and small sized sensory items to target the sensory system during activities including poms, shaker egg, gel exercise balls, and similar items; flat items to develop key pinch to tip pinch (links, magnet discs, etc.); small round items to develop movement from fingers to tips for intricate fine motor skills and finger isolation to work with small buttons, toothpaste caps, grooming, writing, pointing, including items such as beads, nuts and bolts, centimeter cubes, stringing rings, and similar items; active participation tools including tracing cards, timers, finger fidgets balls to hold, roll or throw, squeeze toys, putty, stamps, klixx, and similar items; items for eye hand coordination activities including pop tubes with marbles, pegboard with pegs and poms on top, wiggle jiggle pen, and similar items; tools to allow for patterns, sorting, and counting, including low level and high level and including rainbow caps, centimeter cubes, klixx and similar items; tools related to activities to regain job skills such as putting items together (machinery, furniture, cooking, cashier, etc.) nuts and bolts, pushing bolts into putty for strength and agility, counting or sorting links, centimeter cubes, beads, rainbow caps, poms, and counting magnet discs as change; lacing tools including large and small laces, flexible and stiff laces, smooth to furry laces including laces relate directly to development for tying shoes, dressing, IADLs, sewing, lacing, and other similar activities.

In some embodiments, the therapeutic tools can be carefully selected and researched to meet development needs and to promote success in regaining an independent level of function. In other embodiments, the activities can be carefully selected and researched to meet developmental needs and promote success in regaining an independent level of function. In yet other embodiments, multiple activities can correspond to an individual therapeutic tool. In some embodiments, the multiple activities that correspond to an individual therapeutic tool can be divided into levels. In other embodiments, the multiple activities that correspond to an individual therapeutic tool can be divided into three embodiments. In yet other embodiments, a subject can begin with a level one activity with an individual therapeutic tool. In some embodiments, once the subject has achieved the Level 1 activities, the subject can work with the Level 2 activities and then move to the Level 3 activities. In some embodiments, a subject can begin with either Level 2 or Level 3 activities

In some embodiments, Level 1 can comprise the therapeutic tools and activities shown in Table 1.

TABLE 1

| Therapeutic tool | Activity |
|---|---|
| Beads | Improve tripod and/or scissors grasp by sorting by color and shape and |
| | picking up using progressive grasping pattern. Use Bendeez or tongs to |
| | pick up beads and place in container. |
| Bendeez | Strengthen hands by bending into a variety of shapes such as a circle, an |
| | animal, or a letter. After each activity reshape into a straight line and |
| | bend in the middle to use as tongs to pick up items and strengthen tripod |
| | grasp. |
| Block Crayons | Improve tripod grasp by beginning with finger hold on the largest part of |
| | the crayon. Use to stack into a tower. |
| Centimeter Cubes | Improve visual awareness and pincer grasp by pulling apart stacks and |
| | putting back together using visual awareness to line up squares |
| | symmetrically. |
| Erasers | Improve development of tripod and pincer grasp by pinching the flat ends |
| | with tripod grasp and using one in each hand to pick up a large wooden |
| | cube by simultaneously squeezing the block between the erasers. Place |
| | erasers on flat surface and pick up using scizzors. Change hands and |
| | complete on the opposite side. |
| Exercise Tubing | Strengthen upper extremity by exercising elbows, wrists, shoulders, and |
| | finger flexion and extension. Adapt activity to subject's care and |
| | condition. |
| Lacing | Use lace to lace colored rings, large cubes and magnetic rings. Use nylon |
| | rope to tie a bow. Use shoelace to increase difficulty by lacing objects in |
| | patterns or sequences. |
| Magnetic Balls | Pull apart magnetic balls and put back together. Line up and pull across |
| | the table using one leading marble. Place magnetic balls on magnetic |
| | wand, two balls on top and two on the bottom, then change place on wand. |
| Magnetic Discs | Sort circles by color. Make letters or numbers. Start with a half-made |
| | shape and complete the shape for visual closure practice. After |
| | completing, use magnetic wand to pick them up. Alternate right hand and |
| | left hand. |
| Magnetic Rings | Separate by color using tripod grasp, spread out on large surface area, pick |
| | up using one magnet to begin. Pick up by color or in sequential patterns |
| | or by color. Improve supination and pronation of hand by allowing |
| | magnetic force to repel rings and then rotating to the other side to pick up. |
| Magnetic Wand | Attract magnetic balls, discs, and magnetic rings. Pick up by color, make |
| | shapes, and move magnetic balls in circular motion or in line to the left |
| | and right. |
| Marbles | Practice hand coordination by holding several marbles in one hand, roll |
| | one marble at a time up to the thumb and index finger and place back in |
| | the container. Over time, increase amount of marbles held and transfer |
| | from one hand to the other. Flick marbles with index finger and thumb to |
| | a specific destination or goal. Use both right and left hands. |
| Nuts and Bolts | Begin with larger size nut and bolt and improve tripod grasp strength and |
| | endurance by unthreading and threading nut. Progress to smaller nuts and |
| | bolts. |
| Pegs and Pegboard | Place pegs in board using pincer grasp. Rotate by using color groups or fil |
| | outside of board with pegs. Fill inside or play follow-me-around-board |
| | while taking turns. |
| Pencils | Begin with small pencil to promote tripod grasp. While holding pencil |
| | place a magnetic disc in your palm and use the fourth and fifth finger to |
| | keep it in place to promote proper grasp for writing. Progress to larger |
| | pencil. |
| Poms | Pick up poms with pincer grasp and sort into colors or groups. Flick with |
| | finger to a specific goal with index finger and thumb. |
| Pop Tubes | Lengthen tube by pulling with both hands, hold with one hand to stabilize |
| | and pull with opposite hand. Follow same procedure to push back |
| | together. Use right and left hands. Connect tubes for throwing and |
| | catching activities. |
| Rainbow Caps | Stack caps to build a tower, knock it down and flip caps to right side up |
| | using wrist rotation and tripod grasp. Create patterns and sequences using |
| | the colors. Stack and put away by placing tube over stack. |
| Rainbow Links | Improve fine motor skills by using scissor grasp to put together links in |
| | color groups or patterns. Progress from a scissor grasp to a pincer grasp to |
| | link together. |
| Sand Timer | Use to time activities for speed and agility. Use as a visual motivational |
| | tool to complete tasks quickly. Log time and try to increase speed with |
| | each use. |
| Scizzors | Improve scissor skill strength safely and effectively by using proper finger |
| | positioning. Place at downward angle to pick up poms, pegs, and beads. |
| | Use right hand and left hand. |
| Stamps | Improve tripod grasp by stamping design on paper. Use tripod grasp and |
| | proper hand pressure on design. Improve independence by taking lid on |
| | and off, using stabilizing hand while stamping, and improve eye-hand |
| | coordination by putting away in container. |
| Stringing Rings | Sort by color, create patterns, and string. Use as a foundation to hold |
| | marbles and poms. String rings onto pipe cleaners or nylon rope. |
| Theraputty | Improve strength and sensor skills by using both hands to roll out. Start |
| | with yellow putty and progress to pink. Pinch with thumb and index |
| | finger along the snake-like roll to build finger strength in right and left |
| | hand. Hide large objects in putty and then remove from putty. Strengthen |
| | hands by squeezing in right and left hands. |
| Tongs | Improve fine motor skills by using tongs to pick up and manipulate other |
| | therapeutic tools. Begin by picking up larger items such as blocks, poms, |
| | marbles, and erasers. Use right and left hands. Progress to smaller items. |
| | Begin by holding tongs close to end and progress to holding higher up on |
| | the tongs. |
| Tracing Cards | Learn correct writing pressure by placing paper over tracing card, using |
| | large crayon or pencil to cover surface area of card completely by using |
| | vertical lines by rubbing tool back and forth. |
| Wire Wizard | Place index and middle finger on each side of the wizard, press thumb and |
| | hold red button on the end of the wizard to extend prongs. Use right and |
| | left hand to repeat. This is a strategic activity to strengthen thumb |
| | opposition for self-care and writing skills. |
| Wood Blocks | Improve grasping patterns by beginning with raking grasp and progressing |
| | to pincer grasp. Use blocks as a large beginning tool with tongs and |
| | Bendeez. String onto pipe cleaners or laces |

In some embodiments, Level 2 can comprise the therapeutic tools and activities shown in Table 2.

TABLE 2

| Therapeutic tool | Activity |
|---|---|
| Beads | Progress from tripod grasp to a pincer grasp to pick up beads and lace |
| | onto pipe cleaner or shoelace. Pick up beads one at a time and place in |
| | palm. Challenge activity by increasing number of beads in palm. Place |
| | back into container by moving bead from palm to a pincer grasp to put |
| | away one at a time. Do activity with right and left hand. Use Wire |
| | Wizard to pick up beads and place in container or use rainbow cap turned |
| | upside down as a tray. |
| Bendeez | Use as tongs to pick up medium sized items. Hold with pincer grasp at |
| | the end of the Bendeez (as tongs), move hand position to middle to |
| | increase difficulty. Roll into challenging shapes or position using pincer |
| | grasp, palmar grasp, and tripod grasp. |
| Block Crayons | Use individual fingers inside crayons to color and use correct pressure. |
| | Use a tripod grasp on outside tip to gain success in writing and |
| | strengthening grasp. |
| Centimeter Cubes | Perform resistive strengthening exercise by building shapes, numbers, |
| | letter, and increasing difficulty level of copying patterns. Use Bendeez or |
| | tongs to stack or to place in shapes and letter formations. |
| Erasers | Increase strength and coordination of pincer grasp by pinching flat ends of |
| | eraser, one in each hand and using to pick up small items such as |
| | millimeter cubes, pegs, and beads. |
| Exercise Tubing | Tie knot in end and use as a slingshot to launch tubing to a target. Use for |
| | upper and lower extremity strengthening. |
| Lacing | Use pipe cleaner to lace larger items and move to small items such as |
| | blocks to small beads. Use nylon rope around an object to practice tying a |
| | shoelace. The object can include a desk leg, a person's arm, or a pencil |
| | holder. Use shoelace as a challenge to lace on blocks and beads. |
| | Shoelace is more flexible and will require more coordination. |
| Magnetic Balls | Place two magnetic balls on a flat surface and then one magnetic ball in |
| | each hand. Place the balls in each hand in the air over the balls below. |
| | Control direction of movement while keeping the balls in the air. Move |
| | the balls back and forth and left to right. Attach all balls on the bottom of |
| | the magnetic wand in a straight suspended line. Stand up and walk with |
| | wand and attached balls to improve coordination and motor control. Place |
| | balls on a desktop and use one ball under the desk to control the balls on |
| | top of the desktop. |
| Magnetic Discs | Improve visual tracking for reading and academics by making complex |
| | patterns; create animals, letters, and shapes. Line discs up in a long |
| | straight line. Use the magnetic wand to pick them up in order from right |
| | to left and left to right. Lay down discs again and repeat. |
| Magnetic Rings | Stand rings up on ends and connect like a train. Place on exercise tubing |
| | for strengthening exercises one at a time. Add more rings to increase |
| | strength and resistance. |
| Magnetic Wand | Lead the magnet balls or attach them to the top and bottom of wand. Lift |
| | all magnets attached to bottom of the wand without dropping during |
| | walking or moving. Use to pick up magnetic rings by color or pattern. |
| | Magnetic discs can be picked up with wand, use as a game to see how |
| | many can be attached to the wand, or to pick up shapes, letters, or other |
| | items. |
| Marbles | Hold in palm, roll one marble up to index finger and place on magnet ring |
| | center or magnetic rings. After marbles are placed remove using pincer |
| | grasp and hold in palm. Increase number held in palm as progress is |
| | made. |
| Nuts and Bolts | Take nuts off the bolts, turn the bolts and set upright on table. Hold with |
| | stabilizing hand and screw nuts back on. Use right and left hand to |
| | practice. |
| Pegs and Pegboard | Place pegs in board. After placing pegs into board place marbles or poms |
| | on top of pegs using pincer grasp. Keep pegs in hand and then roll them to |
| | tip of index finger and thumb to place them. Repeat process to remove |
| | pegs. |
| Pencils | Improve tripod grasp by using small pencil. Progress to larger pencil. |
| Poms | Use tripod grasp to pick up poms and place in palm. Increase amount of |
| | poms in palm. Use right and left hand. Use tactile blowing fish to blow |
| | poms to a target. Play with poms for a tactile sensory experience in |
| | palms, balance on arms, back of hands, etc. |
| Pop Tubes | Lengthen tube by pulling with shoulder extension. Place marble inside |
| | and cover ends with palms. Raise left hand and visually follow marble in |
| | tube from right to left and left to right. Connect the ends and pull to a |
| | circle or square shape and use visual tracking skills to follow marble. |
| Rainbow Caps | Stack using tripod grasp, wire wizard, or Bendeez as tongs. Sort by color |
| | and place in groups. Set up sequence patterns to copy and follow |
| | directions. Use challenging patterns to increase difficulty. To improve |
| | ability to follow verbal directions start with verbal instructions to form |
| | one or two patterns. Follow with more complex verbal instructions. |
| Rainbow Links | Copy patterns or put together while standing, sitting, prone on elbows, or |
| | laying on back. Use to clip to a desk support or small stationary object to |
| | make chains in patterns or by color. |
| Sand Timer | Use as a timer in vertical position to motivate timed academic activities |
| | such as: saying alphabet, counting, building towers, or completing other |
| | activities. Use timer to give boundaries to difficult activities such as; “We |
| | will do this activity for as long as the timer runs, then we will take a |
| | break”. Lengthen the time between starts and stops. |
| Scizzors | Roll Theraputty into long roll (snakelike) and cut indentations using |
| | proper finger position with Scizzors. Use right and left hand. Scizzors |
| | include small spring to assist in opening Scizzors for use. |
| Stamps | To address correct writing pressure remove lids, place one stamp in each |
| | hand, and stamp at the same time. Pay specific attention to the quality of |
| | stamp made: light, half stamp, or dark. Adjust pressure accordingly. |
| Stringing Rings | Create color patterns or sequences to copy. String rings onto nylon rope, |
| | then switch to pipe cleaner, and finally switch to a shoelace. |
| Theraputty | Roll Theraputty into a long roll. Use Scizzors to cut indentation into putty |
| | using right and left hands. Use indentations to pull apart section of putty |
| | and roll into circles using thumb, index finger, and second finger to create |
| | a ball. Complete activity with right and left hands. Hide medium size |
| | objects in putty and then hunt for objects and pull out with pincer grasp. |
| Tongs | To promote supination and pronation of wrist rotate pegs from lying |
| | position to standing on flat end. Repeat by color or by pattern. Use to |
| | place items in container, tube, or tray. Challenge by increasing distance of |
| | reach to container, and by either sitting or standing during activity. |
| Tracing Cards | Place paper over card. Use small pencil to trace design onto paper using a |
| | tripod grasp. Cover completely with vertical or horizontal lines to |
| | complete activity. |
| Wire Wizard | Spread magnetic rings out on surface area. Place wizard in right hand and |
| | use left hand to pick up magnetic rings to place on the white tip of wizard. |
| | Repeat by color or sequence. Switch to left hand and complete activity by |
| | picking up magnets with right hand and placing on wizard. This activity |
| | promotes excellent thumb opposition practice to complete any picking up |
| | activity (counters, beads, links, pegs, poms, marbles, etc.) |
| Wood Blocks | To teach position in space use blocks to copy a three dimensional |
| | structure. Practice recognizing in front of, to the side, on top, etc. String |
| | blocks with a shoelace. |

In some embodiments, Level 3 can comprise the therapeutic tools and activities shown in Table 3.

TABLE 3

| Therapeutic tool | Activity |
|---|---|
| Beads | Pick up circle beads with pincer grasp one at a time by moving beads to |
| | palm. Then use a pincer grasp to place through the slot in the tube lid. |
| | Place bead on its side and use scissors to pick up and place in tube. Place |
| | bead in Theraputty by pressing down with isolated finger for resistive |
| | exercise. Use different fingers such as index, middle, ring, and little finger |
| | on right and left hands. |
| Bendeez | Increase speed and time limits on making animals, alphabet shapes, etc. |
| | Use as tongs to pick up small pony beads and place in container with lid |
| | on to address eye hand coordination and finger strength. |
| Block Crayons | Use a tripod grasp on tip of crayon to practice writing/tracing activities to |
| | increase position and strength. |
| Centimeter Cubes | Use erasers to pinch on flat end with pincer grasp and pick up cubes and |
| | stack to build towers. Excellent tool for copying challenging patterns |
| | while strengthening pincer grasp. Introduce letters or numbers with |
| | colored patterns using the blocks. When the task is completed, arrange |
| | into solid colored rows and return to tubes. |
| Erasers | Hold using flat ends with pincer grasp. Hold one eraser in each hand and |
| | use to pick up millimeter blocks and to stack into a tower. Do this by |
| | pressing the block between the erasers. Manipulate blocks to flat side to |
| | stack and build tower. Decrease time limit to create challenge and |
| | progress. |
| Exercise Tubing | Use the magnetic discs to lace onto the exercise tubing, add disc to |
| | increase weight for strengthening exercises. Consult specialist for specific |
| | exercises. |
| Lacing | Use pipe cleaner and small beads to make patterns for necklaces or |
| | bracelets. Use nylon rope to tie a knot and bow. Use shoelace to lace small |
| | beads or large magnetic discs for increased strength and dexterity. Use |
| | different types of laces to give opportunity for two-handed activities with |
| | light, medium, and heavy tools |
| Magnetic Balls | Hold one ball in air six to twelve inches directly above two balls below. |
| | Use wrist rotation in a circular motion to cause the balls below to spin. |
| | Rotate balls with fast and slow spinning speeds. Use the magnet balls with |
| | magnet rings to build a tower (magnet ring, ball, magnet ring, ball, etc.). |
| Magnetic Discs | Lay discs out on flat surface; flip them over to work on pincer grasp. Use |
| | index finger to push disc through red lid top to address fine motor |
| | coordination and to assist in doing self-care tasks such as fastening |
| | clothing buttons. Stack discs by color or sequence to make tower. |
| Magnetic Rings | Place rings on hard end tip of wire wizard or large pencil to gain strength |
| | with tripod grasp. Perform with right hand and left hand. Use as a base |
| | for pincer grasp activities with marbles, pegs, or poms. Use positive and |
| | negative charge to move, rotate, or spin magnetic rings while holding |
| | magnets in each hand and having two or three together in the middle. |
| Magnetic Wand | Place magnetic discs in long horizontal line. Use wand to pick up discs |
| | from left to right to improve visual tracking. Repeat from right to left. Use |
| | the discs to make letters, animals, shapes, and pick up with magnetic |
| | wand. Place shaker egg on top of magnetic wand to address balance and |
| | help with transition from one event to the other. Use wand to manipulate |
| | and spin magnetic discs and marbles. |
| Marbles | Place pegs in pegboard using pincer grasp. Place marbles on top of pegs |
| | and hold marbles in one palm while placing and gathering marbles. |
| | Increase number of marbles in palm to promote challenge. Hide marbles |
| | in Theraputty and then dig out the marbles with index finger. Also use |
| | marbles to stack with lacing rings to create several three marble towers. |
| Nuts and Bolts | Use nuts and bolts to press indentation into Theraputty. Start with yellow |
| | Theraputty and then move to pink Theraputty to increase resistance. Hide |
| | nuts and bolts in Theraputty and then dig them out. |
| Pegs and Pegboard | Use tongs or Scizzors to rotate the pegs and stand them on end. Use right |
| | and left hands. Press pegs into Theraputty to build resistance and |
| | strength. Play game listed on the pegboard to improve motor planning and |
| | processing. |
| Pencils | When using writing tools watch for tripod position hold. Hold closer to |
| | the tip of the pencil to help hold position and achieve writing success |
| Poms | Place poms on pegs in pegboard or place poms on the pegboard holes. Use |
| | Scizzors, wire wizard, or Bendeez tongs to pick up poms and place in |
| | container and sort into colors. Use with spikey fish to blow poms across |
| | table or to a goal. Increase time and amount limit to promote challenge |
| | and fine motor strength |
| Pop Tubes | Extend tubes out into full open position. Swing in circles in front of body |
| | or over head to create whistle sound. Use one in each hand and spin using |
| | wrist rotation to create double whistle and upper extremity strengthening |
| | exercises. |
| Rainbow Caps | Turn upside down using tripod grasp and then use cap as a tray. Match |
| | cap to same color bead and place in matching cap tray using tripod grasp |
| | or wire wizard. When finished flip all caps right side up and stack into a |
| | tower and place tube on top to complete activity. Place beads back into |
| | container using pincer grasp. |
| Rainbow Links | Make chain using patterns or colors. Then add five links on each link to |
| | increase difficulty and strength with right and left hands. Use one link in |
| | palm and move to pincer grasp position using fine motor coordination to |
| | address in hand manipulation. Place in tube to complete activity using the |
| | right and left hands. |
| Sand Timer | When activity has been successfully achieved during time limit, change |
| | activity or move onto next level. |
| Scizzors | Put pegs in Theraputty, add resistance by pulling pegs out of putty with |
| | Scizzors. Improve visual acuity and coordination by using Scizzors to take |
| | poms off pegboard. Use the Scizzors to pick up items and place back in |
| | small container. Place container in variety of positions: above, down, on |
| | the floor, to the right, and to the left. These activities will promote hand |
| | manipulation and position in space. |
| Stamps | Stack stamps on top of each other after each use. Use to make designs |
| | such as letters, numbers, name, etc. to address strength and writing |
| | pressure. |
| Stringing Rings | Build a tower by stacking the rings on top of each other by colors or by |
| | pattern. To challenge, add marbles to tower such as: ring, marble, ring, |
| | marble, etc. Use right and left hands. |
| Theraputty | Use putty to push and pull for strengthening upper extremities. Hold with |
| | both hands in vertical position and pull putty to make long stretch from |
| | top to bottom. Hold putty horizontally and pull to side for extension |
| | activities. Use finger digits to pull and push into variety of shapes, |
| | animals, letters, numbers, etc. Flatten putty onto surface, hide small |
| | objects in putty and roll up into a ball. Dig out beads or stringing rings |
| | with pincer grasp using right and left hands. |
| Tongs | Use Tongs to manipulate small objects such as: millimeter cubes, |
| | stringing rings, and small beads. Place small items in Theraputty and |
| | slightly press into putty. Use tongs to pull out of putty which as a |
| | strengthening resistive exercise. Use to manipulate small items into |
| | shapes, letters, or numbers. These activities will aid in self-help skills such |
| | as zippers, toothpaste lids, etc. |
| Tracing Cards | Use outside square as a stencil to trace shapes. Then add tracing design in |
| | the center using a tripod grasp on writing tool. Use crayon tip or pencil to |
| | complete activity |
| Wire Wizard | Use thumb opposition movements to open and close. Use to pick up |
| | medium sized items such as: links, math counters, and animal beads. Use |
| | right and left hands. This activity will effectively address writing tool |
| | pressure, finger strength, hand manipulation, and coordination. |
| Wood Blocks | Lace blocks with pipe cleaner. Repeat with a shoelace. Repeat using |
| | patterns and/or colors. |

In some embodiments, a sensory level can comprise the therapeutic tools and activities shown in Table 4.

| Therapeutic tool | Activity |
|---|---|
| Blow Fish | Great beginner sensory toy. Use to move a pom across the table. Put air |
| | on face, hands, etc. May use in palm in beginning, then progress to tripod |
| | squeeze (thumb, index and second finger). |
| Egg Shaker | This toy is magnetic and can be used for transition with the magnetic |
| | wand. Also use to wake up sensory system. Dance and shake, catch from |
| | one hand to another, begin with shaking in palm and move to using finger |
| | tips to shake. |
| Finger Spring | Fold in each end. Use index finger on the right hand on one side and index |
| | finger on the left hand on one side and push toward the center |
| | simultaneously. Repeat with middle fingers and so on for strengthening |
| | exercises. Also try placing thumb on the bottom and index finger on the |
| | top. Press fingers together simultaneously to exercise fingers. Change to |
| | thumb and middle finger and so on. Use repetitions for gain strength and |
| | coordination |
| Flying Frogs | Use index fingers, one in the front and one in the back, to fling frog and |
| | make it fly. Make a game of launching frogs into bucket or to a goal. Face |
| | palms toward each other. Place flying frog loops on index fingers of both |
| | hands. Pull apart simultaneously for hand strengthening exercises. Change |
| | from index finger to other fingers and use repetition to increase strength. |
| Gel Exercise Ball | This soft squeeze ball is used with both right and left hands. Squeeze and |
| | hold to build strength. |
| Hand Massager/Top | This tool is fun to roll on arms, fingers, and in hands. Use to wake up |
| | hands for activity. Use as a spinning top with right and left hands. |
| Jiggler | Calming vibration will alert and wake up sensory system. Good vibration |
| | for hands, arms, and cheeks. May be used as an oral motor tool. Refer to |
| | specialist for specific instructions. |
| Jumping Frog | Use individual finger to make bright durable frog jump and flip. Use |
| | index finger to flip the frog with right and left hands, progress to other |
| | fingers. To create challenges do the following: measure distance, jump to |
| | the left and to the right, jump to a goal or inside circle (decrease size of |
| | goal over time), and jump over smaller objects. |
| Klixx | Challenging two handed activity. Manipulate shapes by pulling apart and |
| | pushing back together. Make a picture frame, animals, or alphabet letters. |
| Liquid Timer | This mesmerizing tool can be used as motivation to complete activities |
| | with speed and coordination. Good for sensory stimulation, transition, or |
| | calming. |
| Magic Wire Loop | Pull and push into different shapes and designs. Use visual closure to |
| | complete ball shape, basket, etc. by matching the top shape to the bottom. |
| | Use for transition or calming. |
| Net Squeeze Toy | Irresistible tool for the hands. Squeeze with both right and left hands. This |
| | tool will help wake up the sensory system, use with supervision. Do not |
| | place in mouth. |
| Smiley Stress Ball | Smile and squeeze with repetition on right and left hand. Hold hair with |
| | pincer grasp and bounce. |
| Spike Ball/Finger | Perfect sensory seeking tactile ball and massager. Roll in hands and on |
| massager | arms. Use fine motor skills to twist open. Use the finger massager on each |
| | finger to prepare for fine motor tasks or writing by rolling up and down |
| | each finger. |

FIG. 3AB illustrates some embodiments of a slotted cap. In other embodiments, the slotted cap can comprise a slot that can be used as a therapeutic tool. Disks can be placed through the slot to build targeted skills. In yet other embodiments, the slotted cap can further comprise a round cutout that can be used as a therapeutic tool. Round bead or similar sized therapeutic tools can be placed through the round cutout to build targeted skills. In some embodiments, the slotted cap can be detachably fitted to a tube container. In other embodiments, the slotted cap can retain items in the tube container. In yet other embodiments, the slotted cap detachably fitted to a tube container can be used as a therapeutic tool as described above.

FIG. 3AC illustrates some embodiments of stringing balls that can be used as a therapeutic tool. In some embodiments, the stringing balls can be used as therapeutic tools in similar fashion as described above for beads, centimeter cubes, lacing, magnetic balls, marbles, and other similar items.

FIG. 4O illustrates some embodiments of a gel bead pack. In some embodiments, the gel bead pack can be used as a therapeutic tool. In other embodiments, the gel bead pack can be used as a therapeutic tool as described above for bendeez, theraputty, and similar items. In some embodiments, the gel bead pack can be used to improve finger and hand strength. In yet other embodiments, the gel bead pack can be used as a sensory tool. In some embodiments, the gel bead pack can be used in similar fashion as described above for gel exercise ball, net squeeze tool, smiley stress ball, spike ball, and similar items.

FIG. 4P illustrates some embodiments of a gel squeeze toy. In some embodiments, the gel squeeze toy can be used as a therapeutic tool. In other embodiments, the gel squeeze toy can be used as a therapeutic tool as described above for bendeez, theraputty, and similar items. In some embodiments, the gel squeeze toy can be used to improve finger and hand strength. In yet other embodiments, the gel squeeze toy can be used as a sensory tool. In some embodiments, the gel squeeze toy can be used in similar fashion as described above for gel exercise ball, net squeeze tool, smiley stress ball, spike ball, and similar items.

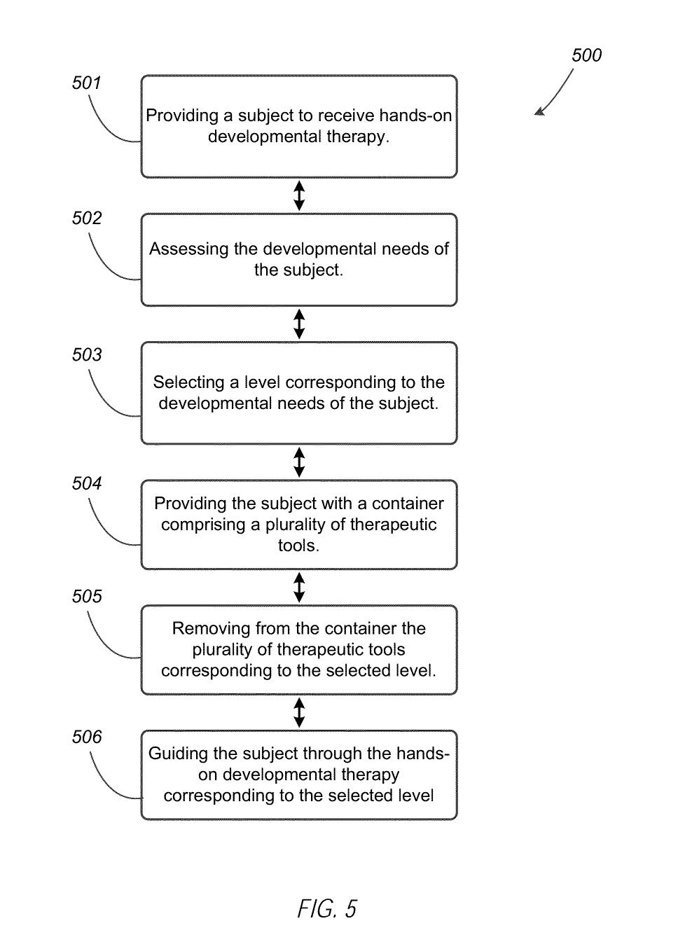

FIG. 5 illustrates some embodiments of method 500 of hands-on developmental therapy. In some embodiments, method 500 can comprise methods of providing hands-on developmental therapy to a subject by a care provider. In other embodiments, the method 500 can comprise providing a subject to receive hands-on therapy 501. In yet other embodiments, the method 500 can comprise assessing the developmental needs of the subject 502. In some embodiments, the method 500 can comprise selecting a level corresponding to the developmental needs of the subject 503. In other embodiments, the method 500 can comprise providing the subject with a container comprising a plurality of therapeutic tools 504. In yet other embodiments, the method 500 can comprise removing from the container the plurality of therapeutic tools corresponding to the selected level 505. In some embodiments, the method 500 can comprise guiding the subject through the hands-on therapy corresponding to the selected level 506.

In some embodiments, a method of providing hands-on developmental therapy to a subject by a care provider can comprise: providing a subject to receive hands-on therapy; assessing developmental needs of the subject; selecting a level corresponding to the developmental needs of the subject; providing the subject with a container comprising a plurality of therapeutic tools; selecting from the container the plurality of therapeutic tools corresponding to the selected level; and guiding the subject through the hands-on therapy corresponding to the selected level. In other embodiments, the method can further comprise a subject suffering from a developmental disorder. In yet other embodiments, the developmental disorder can include one or more of Down's Syndrome, autism, ADD/ADHD, dyslexia, developmental delay, Fragile-X syndrome, sensory processing disorder, sensory integration dysfunction, learning disability, cognitive disability, or cerebral palsy. In some embodiments, the hands-on therapy can comprise a therapy program administered by a therapist. In other embodiments, the therapist can include one or more of a physical therapist, an occupational therapist, or a speech and language therapist. In yet other embodiments, the hands-on therapy can be provided in the subject's home between visits to a therapist. In other embodiments, the method can further comprises levels corresponding to the levels of Tables 1, 2, and 3, wherein each level comprises activities of increasing difficulty and wherein the subject uses the same plurality of therapeutic tools for each level. In yet other embodiments, the method can further comprise a sensory level corresponding to the level of Table 4, wherein the sensory level comprises therapeutic tools and activities configured to improve the sensory system of the subject.

In some embodiments, the method can further comprise improving fine motor skills, improving strength, improving sensory skills, or combinations thereof. In other embodiments, the hands-on therapy can comprise the subject interacting with the therapeutic tools in developmental activities corresponding to daily living activities. In yet other embodiments, the hands-on therapy can comprise the subject interacting with the therapeutic tools in developmental activities corresponding to academic activities. In some embodiments, the hands-on therapy can comprise the subject interacting with the therapeutic tools in developmental activities corresponding to writing activities. In other embodiments, the hands-on therapy can further comprise the subject interacting with the container.

In some embodiments, a system of hands-on developmental therapy can comprise: a subject requiring hands-on therapy; a container comprising a plurality of therapeutic tools; levels corresponding to Tables 1, 2, 3, and 4, wherein each level comprises activities of increasing difficulty and wherein the subject uses the same plurality of therapeutic tools for each level; and a care provider; wherein the care provider selects the level corresponding to hands-on therapy that is appropriate for the subject, removes from the container the plurality of therapeutic tools, and guides the subject through hands-on therapy corresponding to the selected level. The system can further comprise a subject suffering from a developmental disorder. The developmental disorder can include one or more of Down's syndrome, autism, ADD/ADHD, dyslexia, developmental delay, Fragile-X syndrome, sensory processing disorder, sensory integration dysfunction, learning disability, cognitive disability, or cerebral palsy. The system can further comprise the three levels respectively corresponding to hands-on therapy configured to improve fine motor skills, to improve strength, or to improve sensory skills.

In some embodiments a kit for providing hands-on developmental therapy to a subject by a care provider can comprise: a container comprising three tiers; and a plurality of therapeutic tools with accompanying activities, the therapeutic tools and activities comprising levels of Tables 1, 2, 3, and 4; wherein a care provider assesses developmental needs of the subject, selects the level corresponding to the developmental needs of the subject, removes from the container the plurality of therapeutic tools, and guides the subject through the hands-on therapy corresponding to the selected level. In other embodiments, the kit can further comprise the three levels respectively corresponding to hands-on therapy configured to improve fine motor skills, to improve strength, or to improve sensory skills. In yet other embodiments, the kit further comprises wherein hands-on therapy further comprises the subject interacting with the container.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

It is contemplated that numerical values, as well as other values that are recited herein are modified by the term “about”, whether expressly stated or inherently derived by the discussion of the present disclosure. As used herein, the term “about” defines the numerical boundaries of the modified values so as to include, but not be limited to, tolerances and values up to, and including the numerical value so modified. That is, numerical values can include the actual value that is expressly stated, as well as other values that are, or can be, the decimal, fractional, or other multiple of the actual value indicated, and/or described in the disclosure.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Toybox Treatments is an appealing hands-on experience for anyone to enjoy. Our mission is to give access to therapeutic tools which motivate, engage, and assist individuals to become more independent and have a better quality of life

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of providing hands-on developmental therapy to a subject by a care provider, the method comprising:
providing a subject to receive hands-on therapy;
assessing developmental needs of the subject;
selecting a level corresponding to the developmental needs of the subject;
providing the subject with a container comprising a plurality of therapeutic tools;
selecting from the container the plurality of therapeutic tools corresponding to the selected level; and
guiding the subject through the hands-on therapy corresponding to the selected level.

2. The method of claim 1, wherein the subject suffers from a developmental disorder.

3. The method of claim 2, wherein the developmental disorder includes one or more of Down's Syndrome, autism, ADD/ADHD, dyslexia, developmental delay, Fragile-X syndrome, sensory processing disorder, sensory integration dysfunction, learning disability, cognitive disability, or cerebral palsy.

4. The method of claim 3, wherein the hands-on therapy comprises a therapy program administered by a therapist.

5. The method of claim 4, wherein therapist includes one or more of a physical therapist, an occupational therapist, or a speech and language therapist.

6. The method of claim 5, wherein the hands-on therapy is provided in the subject's home between visits to a therapist.

7. The method of claim 1, further comprising levels corresponding to the levels of Tables 1, 2, and 3, wherein each level comprises activities of increasing difficulty and wherein the subject uses the same plurality of therapeutic tools for each level.

8. The method of claim 7, further comprising a sensory level corresponding to the level of Table 4, wherein the sensory level comprises therapeutic tools and activities configured to improve the sensory system of the subject.

9. The method of claim 1, wherein the hands-on therapy comprises improving fine motor skills, improving strength, improving sensory skills, or combinations thereof.

10. The method of claim 1, wherein the hands-on therapy comprises the subject interacting with the therapeutic tools in developmental activities corresponding to daily living activities.

11. The method of claim 1, wherein the hands-on therapy comprises the subject interacting with the therapeutic tools in developmental activities corresponding to academic activities.

12. The method of claim 1, wherein the hands-on therapy comprises the subject interacting with the therapeutic tools in developmental activities corresponding to writing activities.

13. The method of claim 1, wherein the hands-on therapy further comprises the subject interacting with the container.

14. A system of hands-on developmental therapy comprising:
a subject requiring hands-on therapy;
a container comprising a plurality of therapeutic tools;
levels corresponding to Tables 1, 2, 3, and 4, wherein each level comprises activities of increasing difficulty and wherein the subject uses the same plurality of therapeutic tools for each level; and
a care provider;
wherein the care provider selects the level corresponding to hands-on therapy that is appropriate for the subject, removes from the container the plurality of therapeutic tools, and guides the subject through hands-on therapy corresponding to the selected level.

15. The system of claim 14, wherein the subject suffers from a developmental disorder.

16. The system of claim 15, wherein the developmental disorder includes one or more of Down's Syndrome, autism, ADD/ADHD, dyslexia, developmental delay, Fragile-X syndrome, sensory processing disorder, sensory integration dysfunction, learning disability, cognitive disability, or cerebral palsy.

17. The system of claim 14, wherein the three levels respectively correspond to hands-on therapy configured to improve fine motor skills, to improve strength, or to improve sensory skills.

18. A kit for providing hands-on developmental therapy to a subject by a care provider, the kit comprising:
a container comprising three tiers; and
a plurality of therapeutic tools with accompanying activities, the therapeutic tools and activities comprising levels of Tables 1, 2, 3, and 4;
wherein a care provider assesses developmental needs of the subject, selects the level corresponding to the developmental needs of the subject, removes from the container the plurality of therapeutic tools, and guides the subject through the hands-on therapy corresponding to the selected level.

19. The kit of claim 18, wherein the three levels respectively correspond to hands-on therapy configured to improve fine motor skills, to improve strength, or to improve sensory skills.

20. The kit of claim 18, wherein hands-on therapy further comprises the subject interacting with the container.
